(19) United States
(12) Patent Application Publication
(10) Pub. No.: US 2024/0408109 A1
(43) Pub. Date: Dec. 12, 2024

(54) PHARMACEUTICAL COMPOSITION COMPRISING C21 STEROID SAPONIN AND USE THEREOF

(71) Applicant: JINAN UNIVERSITY, Guangzhou (CN)

(72) Inventors: Zuocheng QIU, Guangzhou (CN); Jiaxu CHEN, Guangzhou (CN); Ziling TANG, Guangzhou (CN); Qianqian PANG, Guangzhou (CN); Xiaosan LI, Guangzhou (CN); Li YAN, Guangzhou (CN)

(21) Appl. No.: 18/702,818

(22) PCT Filed: Oct. 17, 2022
(86) PCT No.: PCT/CN2022/125749

§ 371 Date: Apr. 19, 2024

(30) Foreign Application Priority Data
Oct. 20, 2021 (CN) CN202111223046.6

(51) Int. Cl. A61K 31/573; A61K 31/58; A61P 25/28

(52) U.S. Cl. CPC A61K 31/573; A61K 31/58; A61P 25/28

(57) ABSTRACT

The present invention relates to a pharmaceutical composition for preventing and/or treating neurodegenerative diseases. On the one hand, the active ingredient C21 steroid saponin of the pharmaceutical composition can significantly reduce the generation of an amyloid β-protein in N2a-APP695 cells and increase the clearance of excess A3; and on the other hand, the active ingredient C21 steroid saponin of the pharmaceutical composition can promote the proliferation of neuron cells, and has a protective effect on the neuronal cytotoxicity induced by neurotransmitters such as glutamate caused by the excessive accumulation of A3, thereby comprehensively playing a role in the treatment of neurodegenerative diseases. Therefore, the pharmaceutical composition has good application prospects in the preparation of an A3 formation inhibitor and a neuroprotective agent, and in drugs for preventing or treating neurodegenerative diseases such as Alzheimer's disease, Parkinson's disease and Huntington's disease.

### TECHNICAL FIELD

The present disclosure belongs to the field of medicine, and relates to a pharmaceutical composition comprising a C21 steroidal saponin and a use thereof. Specifically, the present disclosure relates to a use of a pharmaceutical composition comprising a C21 steroidal saponin in preparation of an amyloid β-protein (Aβ)-formation inhibitor and a neuroprotective agent.

### BACKGROUND

The nerve cell damage is one of the main causes of neurodegenerative diseases such as Alzheimer's disease (AD), Parkinson's disease, and Huntington's disease. The death of nerve cells can lead to cognitive, learning, and memory dysfunctions in patients with neurodegenerative diseases. The oxidative stress response caused by neurotoxic substances such as Aβ or neurotransmitters is regarded as the main cause of nerve cell death. Glutamate is a major endogenous neurotransmitter of central nervous systems, but glutamate at a high concentration can cause the neurofibrillary tangles (NFTs) and the necrosis of neuronal cells in the brain, thereby resulting in cognitive dysfunction. Moreover, the extracellular glutamate toxicity can reduce the uptake of cells for cysteine by damaging the cystine/glutamate antiporter to cause the depletion of glutathione as an intracellular antioxidant. The imbalances in antioxidant levels, such as introduction of calcium ions, generation of reactive oxygen species (ROS) in cells, and lipoxygenase (LOX)-dependent lipid peroxidation, can also accelerate a series of downstream processes causing the death of nerve cells.

Amyloid plaques produced due to the deposition of neurotoxic Aβ are one of the major causes of AD. This is mainly because β-secretase hydrolyzes β-amyloid precursor protein (β-APP) to produce C-terminal fragment-β (CTF-β) and the CTF-β is further hydrolyzed by γ-secretase to produce 40 or 42 abnormally-folded Aβ peptides. The production and abnormal aggregation of Aβ are crucial for the occurrence of AD. Therefore, inhibiting the production of Aβ and increasing the clearance rate of Aβ may be potential therapeutic strategies to delay the progression of AD.

Cynanchum otophyllum Schneid is a common ethnic medicine widely distributed in southwest China, Hunan, Guangxi, and Tibet. It belongs to the Cynanchum genus of the Asclepiadaceae family and is first recorded in Illustrated Catalogue of Plants. Cynanchum otophyllum Schneid is also known as Qingyangshen, and the root of Cynanchum otophyllum Schneid is known as Radix Cynanchi Auriculati in the folk in Lijiang of Yunnan Province. Cynanchum otophyllum Schneid is slightly mild, sweet, and slightly bitter. According to the “Chinese Herbal Medicine in Yunnan”, “Chinese National Herbal Medicine Assembly”, and “Yi Medicine Records”, dried rhizomes of Cynanchum otophyllum Schneid can be used for rheumatic arthralgia, kidney deficiency, low back pain, lumbar muscle strain, traumatic injuries, food stagnation, abdominal swelling and pain, malnutrition in children, and snake and dog bites. Modern pharmacological studies have shown that Cynanchum otophyllum Schneid has anticonvulsant, antiepileptic, antidepressant, sedative, analgesic, immunomodulatory, anti-hepatitis, and anti-Meniere's syndrome effects.

Studies on the chemical composition of Cynanchum otophyllum Schneid have shown that C21 steroidal saponins abundant in this plant are its representative and main pharmacologically active components. However, most of the C21 steroidal saponins are embedded in glucosides and can hardly be separated, and thus only a few types of free C21 steroidal saponins have been discovered. In recent years, studies have shown that the C21 steroidal saponins have an anti-liver fibrosis activity and an anti-epileptic activity, and exhibit strong anti-proliferative activities for various human tumor cell lines. However, there has been no report on neuroprotective activities and anti-Aβ activities of C21 steroidal saponins.

Therefore, the development of a compound with a neuroprotective activity and an anti-Aβ activity against toxic damages of hippocampal neurons is of important clinical significance for the prevention and treatment of neurodegenerative diseases such as AD, Parkinson's disease, and Huntington's disease.

### SUMMARY

In order to solve the problems during the treatment of neurodegenerative diseases in the prior art, the present disclosure aims to provide a pharmaceutical composition for treating a neurodegenerative disease. The pharmaceutical composition can significantly reduce the expression of 3-APP, significantly promote the proliferation of neuronal cells, inhibit the glutamate-induced cytotoxicity, and have a neuroprotective activity for neuronal cells.

To achieve the above objective, the present disclosure is implemented through the following means:

In a first aspect, the present disclosure provides a pharmaceutical composition for preventing and/or treating a neurodegenerative disease, including one or more selected from the group consisting of a C21 steroidal saponin, a pharmaceutically-acceptable salt of the C21 steroidal saponin, and a solvate of the C21 steroidal saponin and a pharmaceutically-acceptable carrier, where the C21 steroidal saponin has a structure shown in the following formula I.

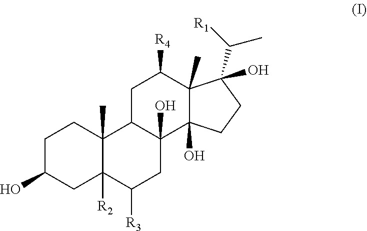

where R₁ is one or more selected from the group consisting of H, O, hydroxyl, optionally-substituted C₁-C₄ alkyl, optionally-substituted C₁-C₄ alkoxy, and

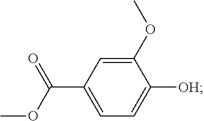

R₂ is one or more selected from the group consisting of H, O, hydroxyl, optionally-substituted C₁-C₄ alkyl, and optionally-substituted C₁-C₄ alkoxy;
R₃ is one or more selected from the group consisting of H, O, hydroxyl, optionally-substituted C₁-C₄ alkyl, and optionally-substituted C₁-C₄ alkoxy;
R₄ is one or more selected from the group consisting of H, O, hydroxyl, optionally-substituted C₁-C₄ alkyl, optionally-substituted C₁-C₄ alkoxy,

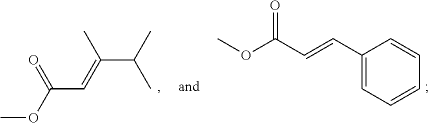

and
adjacent carbon atoms in a multi-membered ring of a compound with the structure shown in the formula I are linked through a single, double, or triple bond.

Preferably, the neurodegenerative disease includes one or more selected from the group consisting of dementia, Parkinson's disease, and Huntington's disease.

Preferably, the dementia includes one or more selected from the group consisting of AD, vascular dementia, Lewy body dementia, and frontotemporal dementia.

Preferably, the pharmaceutically-acceptable carrier includes one or more selected from the group consisting of a filler, an adhesive, a disintegrant, a solvent, a preservative, a lubricant, and a corrigent.

Preferably, the C21 steroidal saponin is one or more selected from the group consisting of compounds 1 to 5 shown in the following structures:

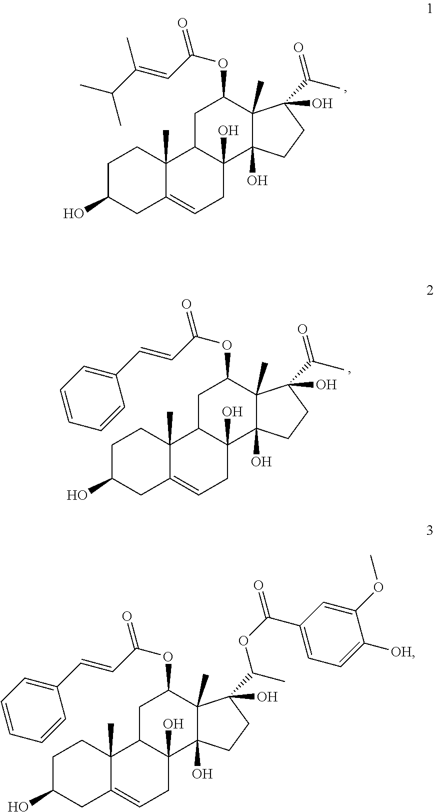

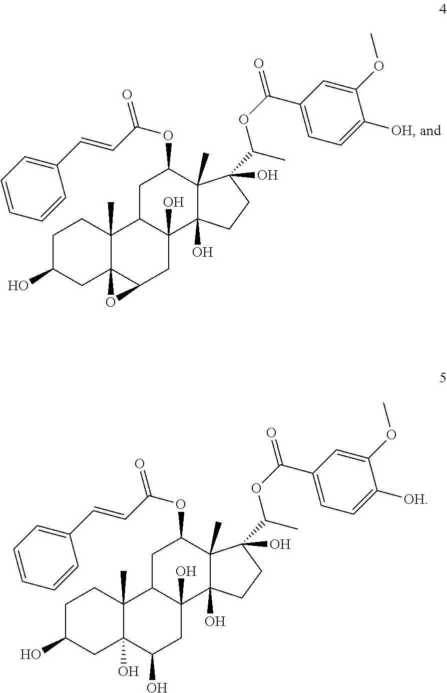

In a second aspect, the present disclosure provides a use of one or more selected from the group consisting of a C21 steroidal saponin, a pharmaceutically-acceptable salt of the C21 steroidal saponin, and a solvate of the C21 steroidal saponin in preparation of a product for preventing and/or treating a neurodegenerative disease, where the C21 steroidal saponin has a structure shown in the following formula I.

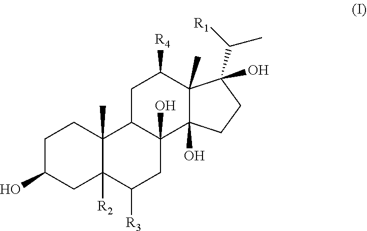

where R₁ is one or more selected from the group consisting of H, O, hydroxyl, optionally-substituted C₁-C₄ alkyl, optionally-substituted C₁-C₄ alkoxy, and

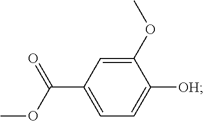

R₂ is one or more selected from the group consisting of H, O, hydroxyl, optionally-substituted C₁-C₄ alkyl, and optionally-substituted C₁-C₄ alkoxy;
R₃ is one or more selected from the group consisting of H, O, hydroxyl, optionally-substituted C₁-C₄ alkyl, and optionally-substituted C₁-C₄ alkoxy;
R₄ is one or more selected from the group consisting of H, O, hydroxyl, optionally-substituted C₁-C₄ alkyl, optionally-substituted C₁-C₄ alkoxy,

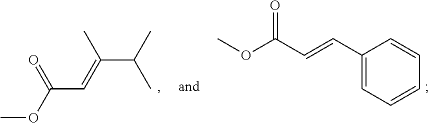

and

adjacent carbon atoms in a multi-membered ring of a compound with the structure shown in the formula I are linked through a single, double, or triple bond.

Preferably, the neurodegenerative disease includes one or more selected from the group consisting of dementia, Parkinson's disease, and Huntington's disease.

Preferably, the dementia includes one or more selected from the group consisting of AD, vascular dementia, Lewy body dementia, and frontotemporal dementia.

Preferably, the C21 steroidal saponin is one or more selected from the group consisting of compounds 1 to 5 shown in the following structures:

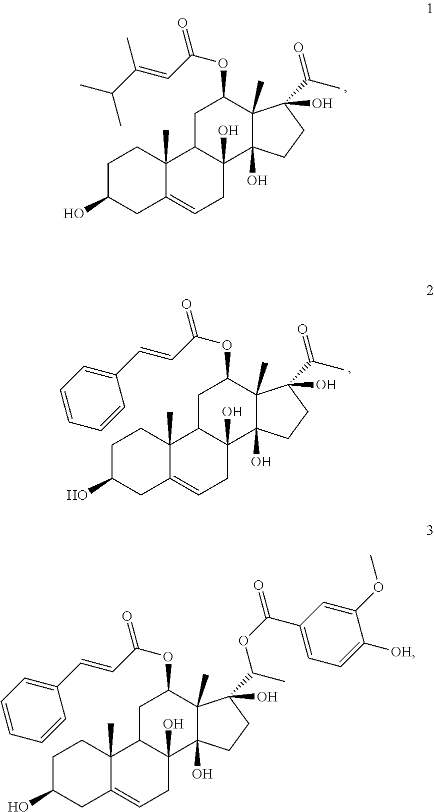

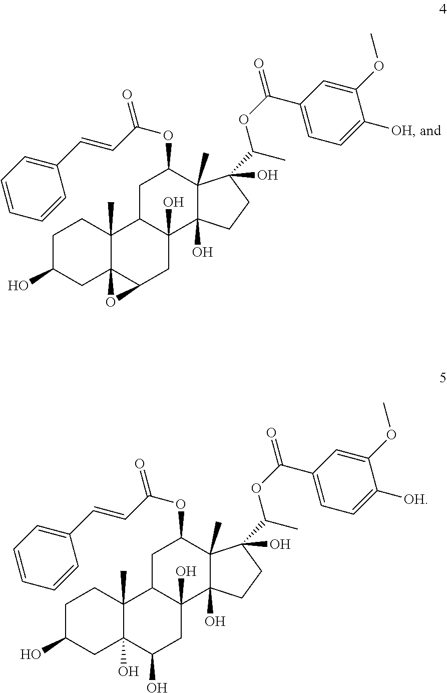

Preferably, the product includes one or more selected from the group consisting of a drug, a health care product, and a food.

With regard to the production of Aβ, existing studies have shown that the inhibition of activities of β-secretase (BACE1) and γ-secretase can reduce the production of Aβ. However, in addition to being involved in the degradation of APP, γ-secretase is involved in the metabolism of many other essential proteins in the body, such as Notch, CD44, E-/N-/P-cadherin, and low-density lipoprotein receptor-related proteins (LRPs), and thus the blocking of the activity of γ-secretase may cause other unexpected serious side effects. For example, the gene knockout of presenilin-1 (PS1) (a core component of a γ-secretase complex) can lead to the death of embryonic mice. Therefore, the impact of the C21 steroidal saponin on BACE1 is investigated in the present disclosure.

With regard to the clearance of Aβ, the degradation of extracellular Aβ is mainly completed by insulin-degrading enzymes (IDEs) and neprilysin (NEP), while the degradation of intracellular Aβ is mainly conducted in lysosomes. The content of Aβ in neuronal lysosomes is very low under physiological conditions, but significantly increases under pathological conditions. The progression of AD is accompanied by a dysregulation of a lysosome system, and the aggregation of Aβ in the lysosome is one of the pathological features of AD.

Recent studies have shown that defects of an autophagy-lysosomal system during AD may precede the generation of Aβ or NFTs, and can impair the function of removing discarded proteins or organelles to aggravate a pathological process of AD. The autophagy is a degradation pathway co-mediated by vesicles and lysosomes, and is very important for protein homeostasis and cellular environments. At an early stage of AD, the autophagy plays an important role in the production and metabolism of Aβ. Although the extracellular aggregates (amyloid plaques) of Aβ and the abnormal phosphorylation of Tau proteins within neurons are obvious pathological markers, defects in the autophagy-lysosomal pathway may precede the generation of these pathological markers.

An autophagy disorder is an important mechanism of excessive accumulation of Aβ: In a normal physiological state, a trace amount of Aβ produced in cells can activate the autophagy by inhibiting mTOR. Because autophagosomes are generated around axons and lysosomes are mainly located around nuclei, autophagosomes are delivered reversely to cell bodies through microtubule systems of axons, bind to lysosomes, and degrade Aβ, such that the production of Aβ and the degradation of Aβ are balanced. In a pathological state, the autophagy in a brain of an AD patient is activated, and autophagosomes increase and aggregate. The aggregated autophagosomes are rich in APP, Aβ, and β- and γ-secretase complexes. Thus, the abnormal increase of autophagosomes is considered to be a source for the production of Aβ.

Due to factors such as an obstructed autophagic flux or defects of degradation functions of lysosomes, the autophagic degradation is hindered and the clearance of Aβ is reduced. Therefore, the regulation of autophagy to accelerate the clearance of Aβ may become an important target for AD treatment. As a result, impacts of a C21 steroidal saponin on autophagy marker proteins LC3B, P62, and Beclin 1 are further investigated in the present disclosure.

LC3B is the first autophagy marker protein discovered, and is in the two forms of LC3B-I and LC3B-II. In the absence of autophagy, LC3B synthesized in cells is processed into cytoplasm-soluble type I LC3B, and is routinely expressed. When the phagocytosis of autophagosomes occurs, type I LC3B is transformed into type II LC3B located on an intracellular autophagosome membrane, and a content of type II LC3B is proportional to a number of autophagic vacuoles. Therefore, LC3B-II is often used as a marker for intracellular autophagy. The transformation of LC3B, namely, an LC3B-II/LC3B-I ratio, can be detected. An increase in the LC3B-II/LC3B-I ratio indicates an increased autophagy level, and a decrease in the LC3B-II/LC3B-I ratio indicates a decreased autophagy level.

Beclin 1 is a specific gene for autophagy, and plays a key regulatory role in the generation of autophagy. The up-regulation of Beclin 1 can stimulate the generation of autophagy. Beclin 1 is an important molecule involved in the association between autophagy and apoptosis. Beclin 1 regulates the apoptosis mainly by binding to Bcl-2 to inhibit the excessive autophagy.

P62 is a common autolysosome substrate, and a content of P62 is negatively correlated with an autophagy level. P62 is an important bridge between LC3 and an ubiquitination substrate to be degraded. When an autophagic flux is activated, a protein polymer produced from P62 can be degraded by autophagosomes, where P62 binds to a membrane protein LC3/ATG8 of an autophagosome, such that a P62-containing protein polymer is transported to the autophagosome.

Compared with the prior art, the present disclosure has the following beneficial effects:
(1) The C21 steroidal saponin, an active ingredient in the pharmaceutical composition provided in the present disclosure, can significantly down-regulate the expression of APP in N2a cells over-expressing human APP695, increase a clearance rate of excess Aβ, promote the proliferation of neuronal cells, and have a protective effect for neurotransmitter (such as glutamate)-induced neuronal cytotoxicity caused by the excessive accumulation of Aβ, thereby comprehensively playing a role of treating a neurodegenerative disease. Therefore, the pharmaceutical composition has a promising application prospect in the preparation of Aβ-formation inhibitors and neuroprotective agents and drugs for preventing or treating neurodegenerative diseases such as AD, Parkinson's disease, and Huntington's disease.
(2) The active ingredient of the present disclosure can inhibit the glutamate-induced cytotoxicity and exhibit a neuroprotective activity for neuronal cells. Therefore, the serious toxic and side effects caused by the inhibition of a secretase activity can be significantly reduced, resulting in high safety.

### BRIEF DESCRIPTION OF THE DRAWINGS

FIG. 1 is a schematic diagram of experimental results of the promotion of 20-O-VK on the proliferation of HT22 cells;

FIG. 2 is a schematic diagram of results of a neuroprotective effect of 20-O-VK for the glutamate-induced damage to HT22 hippocampal neuronal cells;

FIG. 3 shows cell morphology images of an experiment of an impact of 20-O-VK on the glutamate-induced damage to HT22 hippocampal neuronal cells;

FIG. 4 shows schematic diagrams of flow cytometry results in an experiment of the inhibition of 20-O-VK on the glutamate-induced apoptosis of HT22 cells;

FIG. 5 is a schematic diagram of viable cell percentage results in an experiment of the inhibition of 20-O-VK on the glutamate-induced apoptosis of HT22 cells;

FIG. 6 is a schematic diagram of early apoptotic cell percentage results in an experiment of the inhibition of 20-O-VK on the glutamate-induced apoptosis of HT22 cells;

FIG. 7 is a schematic diagram of impact results of 20-O-VK on a survival rate of N2a-APP695 cells;

FIG. 8 is a schematic diagram of western blot (WB) results of impacts of 20-O-VK on the expression of full-APP and CTF proteins in N2a-APP695 cells;

FIG. 9 is a schematic diagram of quantitative analysis results of the expression of full-APP in N2a-APP695 cells under an action of 20-O-VK;

FIG. 10 is a schematic diagram of quantitative analysis results of the expression of CTF in N2a-APP695 cells under an action of 20-O-VK;

FIG. 11 is a schematic diagram of WB results of an impact of 20-O-VK on the expression of a BACE1 protein in N2a-APP695 cells;

FIG. 12 is a schematic diagram of quantitative analysis results of the expression of BACE1 in N2a-APP695 cells under an action of 20-O-VK;

FIG. 13 is a schematic diagram of WB results of impacts of 20-O-VK on the expression of LC3B-I and LC3B-II proteins in N2a-APP695 cells;

FIG. 14 is a schematic diagram of quantitative analysis results of the expression of LC3B in N2a-APP695 cells under an action of 20-O-VK;

FIG. 15 is a schematic diagram of WB results of impacts of 20-O-VK on the expression of APP-full and CTF proteins in N2a-APP695 cells;

FIG. 16 is a schematic diagram of quantitative analysis results of the expression of APP-full in N2a-APP695 cells under an action of 20-O-VK;

FIG. 17 is a schematic diagram of quantitative analysis results of the expression of CTF in N2a-APP695 cells under an action of 20-O-VK;

FIG. 18 is a schematic diagram of WB results of an impact of 20-O-VK on the expression of a P62 protein in N2a-APP695 cells;

FIG. 19 is a schematic diagram of quantitative analysis results of the expression of P62 in N2a-APP695 cells under an action of 20-O-VK;

FIG. 20 is a schematic diagram of WB results of an impact of 20-O-VK on the expression of a Beclin 1 protein in N2a-APP695 cells; and

FIG. 21 is a schematic diagram of quantitative analysis results of the expression of Beclin 1 in N2a-APP695 cells under an action of 20-O-VK.

### DETAILED DESCRIPTION OF THE EMBODIMENTS

To make the objectives, technical solutions, and effects of the present disclosure clear, the present disclosure is described in further detail with reference to embodiments. It should be understood that the specific embodiments described herein are merely intended to explain the present disclosure, rather than to limit the present disclosure.

Unless otherwise specified, the compounds 1 to 5 listed in the context of the present disclosure are extracted from Cynanchum otophyllum Schneid by a conventional method in the prior art, and structures of the compounds are identified by ¹H nuclear magnetic resonance (NMR) and/or ¹³C NMR. The cell lines including HT22 and N2a used in the context of the present disclosure all are cultivated according to the guidelines of American Type Culture Collection (ATCC). All cell lines are identified by the short tandem repeat analysis of China Center for Type Culture Collection (CCTCC) (Wuhan), tested by a polymerase chain reaction (PCR) assay kit (Shanghai Biothrive Sci) to determine whether there is mycoplasma contamination, and cryopreserved in liquid nitrogen for subsequent experiments. The reagents, consumables, or the like used in the present disclosure are commercially available or prepared according to conventional methods. The experimental methods used in the present disclosure, such as cell cultivation, a cell proliferation experiment, an apoptosis experiment, flow cytometry, and a WB experiment, are conventional methods and techniques in the prior art. The instruments and devices applicable to the present disclosure are commercially available, where a microplate reader is the American BioTEK Synergy H1 Hybrid Multi-Mode Reader, and a flow cytometer is the American BECKMAN CONLTER, CytoFLEX S.

Representative results are selected from biological experimental replicates and presented in the accompanying drawings, and data is presented as mean±standard deviation (SD) as specified in the accompanying drawings. All experiments are repeated at least three times. Data is analyzed using GraphPad Prism 5.0. The t-test or analysis of variance is used to compare a mean difference between two or more groups. p<0.05 is considered to indicate a significant difference.

### Example 1 Extraction and Separation of C21 Steroidal Saponins

(1) 15 kg of a dry root powder of Cynanchum otophyllum Schneid was taken and subjected to extraction with 95% EtOH-H₂O (4×30 L) for 2 h under reflux at 90° C. to obtain 2.25 kg of a crude extract.
(2) The crude extract was dissolved in a MeOH/H₂O (2:1, 30 L) solution including 5% of HCl and allowed to reflux for 3 h to obtain a reaction solution, and a pH of the reaction solution was slowly adjusted with a 10% NaOH solution to 7.0; and then the organic solvent was removed under vacuum to obtain a residue, and the residue was dissolved in H₂O and subjected to extraction with EtOAc (3×30 L) to obtain components (1.48 kg) soluble in EtOAc.
(3) The components soluble in EtOAc obtained in the step (2) were subjected to silica gel column chromatography (CC), where elution was conducted with CH₂Cl₂-MeOH (gradients: 95:1→80:1→60:1→30:1→15:1→10:1→5:1→2:1, v/v, and 5 column volumes per gradient) to obtain the compounds 1 to 5.

The compounds 1 to 5 were identified by ¹H NMR and/or ¹³C NMR. Identification results are as follows:

Compound 1: ¹H NMR (300 MHz, CD₃OD): δ_{H} 2.12 (3H, d(1.2), H-7′), 1.08 (3H, d(6.8), H-6′), 1.08 (3H, d(6.8), H-5′), 2.38 (1H, m, H-4′), 5.55 (1H, s, H-2′), 2.17 (3H, s, H-21), 1.16 (3H, s, H-19), 1.57 (3H, s, H-18), 3.20 (1H, t(8.5), H-17), 1.66 (1H, m, H-16), 2.17 (1H, m, H-16), 1.65 (1H, m, H-15), 1.92 (1H, m, H-15), 4.61 (1H, dd(11.5, 4.2), H-12), 1.69 (1H, m, H-11), 1.86 (1H, m, H-11), 1.52 (1H, m, H-9), 2.20 (2H, m, H-7), 5.33 (1H, br s, H-6), 2.28 (2H, d(7.3), H-4), 3.45 (1H, m, H-3), 1.56 (1H, m, H-2), 1.78 (1H, m, H-2), 1.14 (1H, m, H-1), 1.85 (1H, m, H-1). ¹³C NMR (75 MHz, CD₃OD): δ_{c} 167.4 (C-1′), 114.4 (C-2′), 167.4 (C-3′), 39.8 (C-4′), 21.3 (C-5′), 21.3 (C-6′), 16.7 (C-7′), 39.3 (C-1), 31.7 (C-2), 72.6 (C-3), 42.8 (C-4), 140.9 (C-5), 119.2 (C-6), 35.5 (C-7), 75.2 (C-8), 45.5 (C-9), 38.1 (C-10), 25.5 (C-11), 73.0 (C-12), 56.6 (C-13), 88.4 (C-14), 34.5 (C-15), 22.2 (C-16), 61.3 (C-17), 15.8 (C-18), 18.7 (C-19), 212.6 (C-20), 32.3 (C-21).

Compound 2: ¹H NMR (600 MHz, CD₃OD): δ_{H} 7.34 (1H, m^{e}, H-9′), 7.53 (1H, m^{e}, H-8′), 7.35 (1H, m, H-7′), 7.53 (1H, m^{e}, H-6′), 7.34 (1H, m^{e}, H-5′), 7.06 (1H, d(12.1), H-3′), 5.84 (1H, d(12.1), H-2′), 2.15 (3H, s, H-21), 1.18 (3H, s, H-18), 1.67 (1H, m, H-16), 2.85 (1H, m, H-16), 1.88 (1H, m, H-15), 1.98 (1H, m, H-15), 4.53 (1H, dd(11.5, 4.2), H-12), 1.73 (1H, m, H-11), 1.84 (1H, m, H-11), 1.50 (1H, m, H-9), 2.14 (2H, m, H-7), 5.32 (1H, br s, H-6), 2.29 (2H, d(7.3), H-4), 3.45 (1H, m, H-3), 1.57 (1H, m, H-2), 1.79 (1H, m, H-2), 1.12 (1H, m, H-1), 1.85 (1H, m, H-1). ¹³C NMR (150 MHz, CD₃OD): δ_{c} 166.8 (C-1′), 120.8 (C-2′), 144.5 (C-3′), 136.6 (C-4′), 129.0 (C-5′), 130.6 (C-6′), 129.9 (C-7′), 130.6 (C-8′), 129.0 (C-9′), 39.8 (C-1), 31.7 (C-2), 72.6 (C-3), 42.8 (C-4), 140.7 (C-5), 119.2 (C-6), 35.1 (C-7), 74.9 (C-8), 45.2 (C-9), 38.0 (C-10), 25.0 (C-11), 74.4 (C-12), 58.5 (C-13), 92.9 (C-14), 34.1 (C-15), 33.2 (C-16), 89.9 (C-17), 10.1 (C-18), 18.6 (C-19), 212.0 (C-20), 27.5 (C-21).

Compound 3: ¹H NMR (300 MHz, CD₃OD): δ_{H} 7.63 (1H, dd(8.0, 2.0), H-7″), 6.92 (1H, d(8.0), H-6″), 3.89 (3H, s, H-4″-OMe), 7.63 (1H, d(2.0), H-3″), 7.16 (1H, m^{e}, H-9′), 6.85 (1H, m^{e}, H-8′), 7.19 (1H, m, H-7′), 6.85 (1H, m^{e}, H-6′), 7.16 (1H, m^{e}, H-5′), 6.71 (1H, d(12.1), H-3′), 5.69 (1H, d(12.1), H-2′), 1.29 (3H, d(6.0), H-21), 4.64 (1H, dd(12.6, 6.0), H-20), 1.17 (3H, s, H-19), 1.13 (3H, s, H-18), 1.89 (2H, m, H-16), 1.91 (2H, m, H-15), 4.71 (1H, dd(11.5, 4.2), H-12), 1.62 (1H, m, H-11), 1.85 (1H, m, H-11), 1.47 (1H, m, H-9), 2.12 (2H, m, H-7), 5.32 (1H, br s, H-6), 2.28 (2H, d(7.5), H-4), 3.44 (1H, m, H-3), 1.58 (1H, m, H-2), 1.80 (1H, m, H-2), 1.10 (1H, m, H-1), 1.86 (1H, m, H-1). ¹³C NMR (150 MHz, CD₃OD): δ_{c} 167 (C-1″), 123.4 (C-2″), 114.5 (C-3″), 148.7 (C-4″), 56.5 (C-4″-OMe), 152.8 (C-5″), 115.9 (C-6″), 125.8 (C-7″), 168.2 (C-1′), 122.8 (C-2′), 142.5 (C-3′), 136.8 (C-4′), 128.6 (C-5′), 130.0 (C-6′), 129.3 (C-7′), 130.0 (C-8′), 128.6 (C-9′), 39.8 (C-1), 31.7 (C-2), 72.6 (C-3), 42.8 (C-4), 140.5 (C-5), 119.4 (C-6), 35.1 (C-7), 74.9 (C-8), 44.7 (C-9), 37.9 (C-10), 25.6 (C-11), 75.6 (C-12), 57.6 (C-13), 89.5 (C-14), 34.3 (C-15), 33.9 (C-16), 88.5 (C-17), 10.8 (C-18), 18.7 (C-19), 75.8 (C-20), 15.2 (C-21).

Compound 4: ¹H NMR (400 MHz, CD₃OD): δ_{H} 7.51 (1H, d, J=7.6 Hz, H-7″), 6.74 (1H, d, J=8.3 Hz, H-6″), 6.08 (1H, d, J=15.9 Hz, H-2′), 4.70 (1H, q, J=5.8 Hz, H-20), 3.67 (1H, br s, H-3), 3.57 (3H, s, OCH₃-4″), 1.61 (3H, s, CH₃-18), 1.31 (3H, d, J=6.1 Hz CH₃-21), 1.07 (3H, s, CH₃-19). ¹³C NMR (100 MHz, CD₃OD): δ_{c} 168.0 (C-1′), 167.1 (C-1″), 152.7 (C-5″), 148.6 (C-4″), 145.3 (C-3′), 135.5 (C-4′), 131.3 (C-7′), 129.8 (C-6′), 129.8 (C-6′), 129.2 (C-5′), 129.1 (C-9′), 125.3 (C-7″), 123.0 (C-2″), 119.9 (C-2′), 115.8 (C-6″), 113.9 (C-3″), 89.0 (C-14), 88.2 (C-17), 76.7 (C-20), 75.9 (C-8), 74.8 (C-12), 69.6 (C-3), 66.6 (C-6), 65.4 (C-5), 57.7 (C-13), 56.1 (OMe-4″), 45.1 (C-9), 42.3 (C-4), 39.0 (C-1), 36.8 (C-10), 35.0 (C-7), 32.7 (C-15), 31.5 (C-2), 31.0 (C-16), 26.6 (C-11), 17.8 (C-19), 15.3 (C-21), 11.2 (C-18). ESI-MS m/z 679.5 [M+H]⁺.

Compound 5: ¹H NMR (400 MHz, CD₃OD): δ_{H} 7.53 (1H, d, J=7.6 Hz, H-7″), 6.73 (1H, d, J=8.3 Hz, H-6″), 6.08 (1H, d, J=15.9 Hz, H-2′), 4.73 (1H, q, J=5.8 Hz, H-20), 3.63 (1H, br s, H-3), 3.55 (3H, s, OCH₃-4″), 1.64 (3H, s, CH₃-18), 1.31 (3H, d, J=6.1 Hz CH₃-21), 1.27 (3H, s, CH₃-19). ¹³C NMR (100 MHz, CD₃OD): & 168.0 (C-1′), 167.0 (C-1″), 152.7 (C-5″), 148.6 (C-4″), 145.2 (C-3′), 135.6 (C-4′), 131.3 (C-7′), 129.8 (C-6′), 129.8 (C-6′), 129.1 (C-5′), 129.1 (C-9′), 125.3 (C-7″), 123.0 (C-2″), 119.9 (C-2′), 115.8 (C-6″), 113.9 (C-3″), 89.5 (C-14), 88.5 (C-17), 78.8 (C-8), 75.7 (C-5), 76.0 (C-20), 75.9 (C-12), 77.9 (C-6), 68.1 (C-3), 58.2 (C-13), 56.1 (OMe-4″), 41.0 (C-9), 40.0 (C-4), 39.3 (C-1), 34.7 (C-10), 34.4 (C-16), 34.3 (C-15), 32.9 (C-7), 30.9 (C-2), 25.0 (C-11), 18.2 (C-19), 15.4 (C-21), 11.6 (C-18). ESI-MS m/z 697.4 [M+H]⁺.

### Example 2 Experiment of the Promotion of C21 Steroidal Saponins on the Proliferation of Hippocampal Neuronal Cells

(1) HT22 cells in a logarithmic growth phase were taken and inoculated in a 96-well plate at a density of 5,000 cells/well, where 3 replicate wells were set for each group.
(2) The cells were cultivated for 24 h, then a compound was added at different concentrations, and the cells were further cultivated for 48 h. In groups 1 to 6, the compound 3 (20-O-VK) was added at concentrations of 0.01 μM, 0.1 μM, 0.5 μM, 1 μM, 5 μM, and 10 M, respectively. A group C was a control group, and a same volume of a medium was added in the control group.
(3) After the 48 h of cultivation, a medium including the compound was removed, and then a DMEM medium including 10 μL of CCK-8 was added to each well.
(4) The plate was incubated in a 5% CO₂ and 37° C. incubator for 2 h.
(5) The plate was shaken at room temperature for 15 s, and then the absorbance (OD) of each well was determined at 450 nm with a microplate reader.

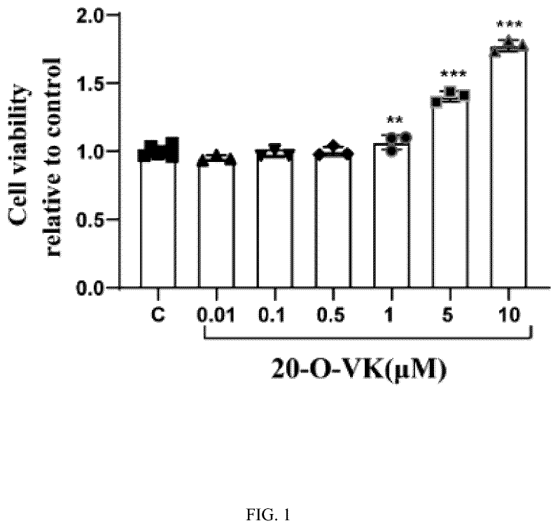

Detection results were shown in FIG. 1. The results show that, in contrast to the group C without a C21 steroidal saponin treatment, in the C21 steroidal saponin treatment groups, a cell proliferation activity increases significantly with the increase of a concentration of the C21 steroidal saponin (1 μM, 5 μM, and 10 μM), and differences between the C21 steroidal saponin treatment groups and the group C are statistically significant (**P<0.01 and ***P<0.001), indicating that the C21 steroidal saponin has a significant activity for promoting the proliferation of HT22 cells.

### Example 3 Neuroprotective Effect of a C21 Steroidal Saponin for the Glutamate-Induced Damage to Hippocampal Neurons

(1) HT22 cells in a logarithmic growth phase were taken and inoculated in a 96-well plate at a density of 5,000 cells/well, where 3 replicate wells were set for each group.
(2) The cells were cultivated for 24 h, then a compound was added at different concentrations, and the cells were further cultivated for 24 h. In groups 1 to 5, the compound 3 was added at concentrations of 0.1 μM, 0.5 μM, 1 μM, 5 μM, and 10 μM, respectively. An NC group was a blank group, and a same volume of a medium was added in the blank group. A group C was a control group, and a same volume of a medium was added in the control group.
(3) After the 24 h of cultivation, 10 mM glutamate was added to each of the groups 1 to 5 and the group C, and cells were further cultivated for 24 h, where no treatment was adopted for the NC group.
(4) After the 24 h of cultivation, a medium including the compound was removed, and then a DMEM medium including 10 μL of CCK-8 was added to each well.
(5) The plate was incubated in a 5% CO₂ and 37° C. incubator for 2 h.
(6) The plate was shaken at room temperature for 15 s, and then the absorbance (OD) of each well was determined with a microplate reader (450 nm).

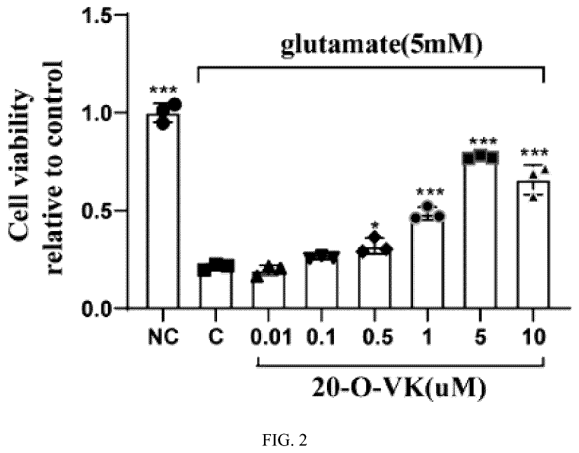

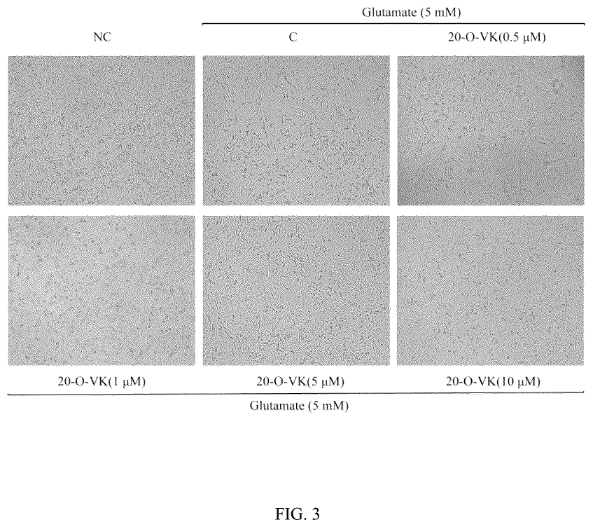

Detection results were shown in FIG. 2 and FIG. 3. The results show that, after a hippocampal neuronal damage model constructed by adding 5 mM glutamate is administered with the C21 steroidal saponin (0.5 μM, 1 μM, 5 μM, and 10 μM), a cell damage rate significantly decreases, indicating that the C21 steroidal saponin has a significant neuroprotective activity for the glutamate-induced damage to hippocampal neurons, and differences between the C21 steroidal saponin treatment groups and the group C are statistically significant (*P<0.05 and ***P<0.001).

### Example 4 Experiment of the Inhibition of a C21 Steroidal Saponin on the Glutamate-Induced Apoptosis of Hippocampal Neurons

(1) HT22 cells in a logarithmic growth phase were taken and inoculated in a 6-well plate at a density of 10⁵ cells/well.
(2) The cells were cultivated for 24 h, then a compound was added at different concentrations, and the cells were further cultivated for 12 h. In groups 1 to 5, the compound 3 (20-O-VK) was added at concentrations of 0.1 μM, 0.5 μM, 1 μM, 5 μM, and 10 μM, respectively. An NC group was a blank group, and a same volume of a medium was added in the blank group. A group C was a control group, and a same volume of a medium was added in the control group.
(3) After the 12 h of cultivation, 10 mM glutamate was added to each of the groups 1 to 5 and the group C, and cells were further cultivated for 24 h, where no treatment was adopted for the NC group.
(4) After the 24 h of cultivation, the conventional apoptosis assay was conducted by a Beckman flow cytometer (CytoFLEX S type) using the Dead Cell Apoptosis Kit with Annexin V Alexa Fluor™ 488 & Propidium Iodide (PI) (Invitrogen, USA).

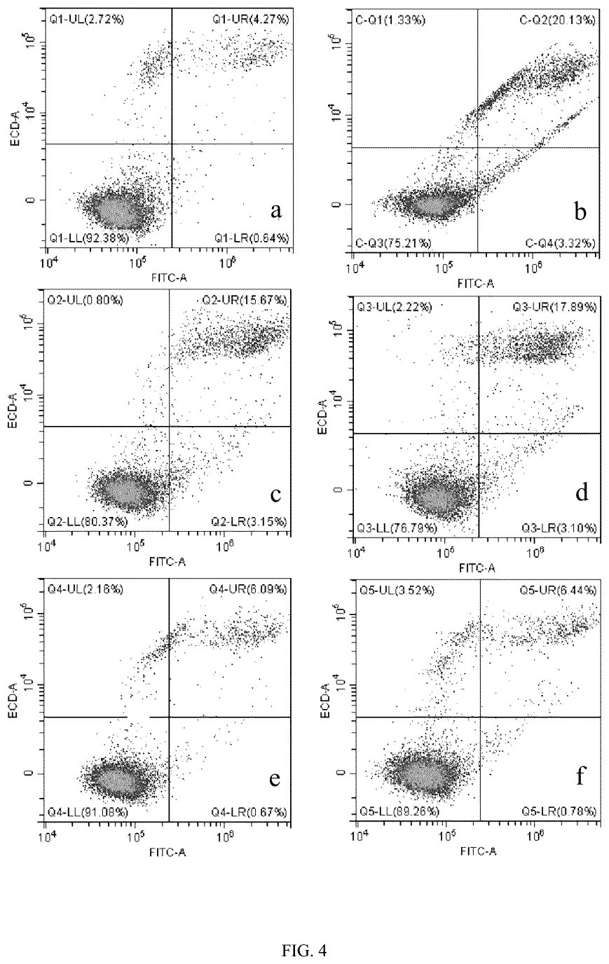

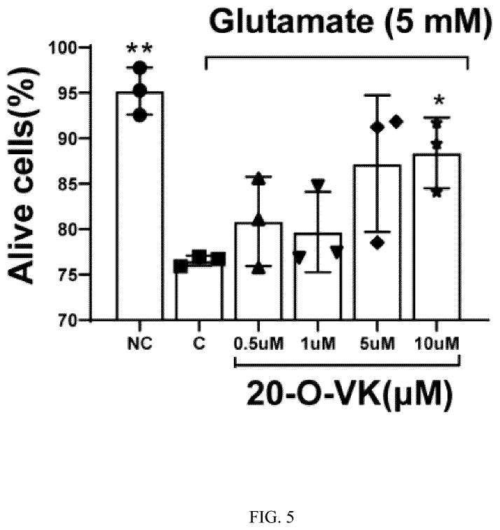

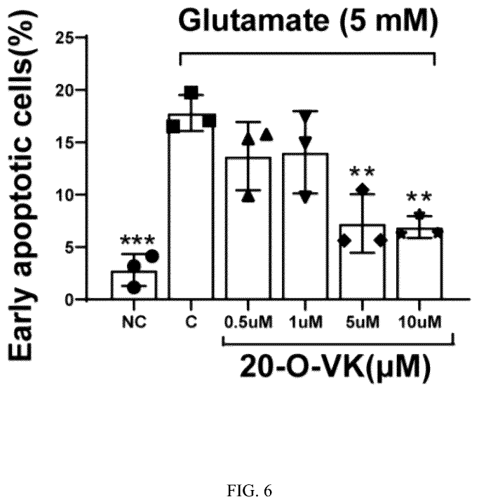

Detection results were shown in FIG. 4 to FIG. 6. The results show that, after a hippocampal neuronal damage model constructed by adding glutamate is administered with the C21 steroidal saponin (5 μM and 10 μM), a cell survival rate significantly increases and a cell apoptosis rate decreases significantly according to flow cytometry assay results, indicating that the C21 steroidal saponin has a significant protective activity for the glutamate-induced apoptosis of hippocampal neurons and can inhibit the apoptosis of hippocampal neuronal cells, and differences between the C21 steroidal saponin treatment groups and the group C are statistically significant (*P<0.05, **P<0.01, and ***P<0.001).

### Example 5 Experiment of an Impact of a C21 Steroidal Saponin on the Proliferation of Neuroblastoma Cells

(1) N2a-APP695 cells in a logarithmic growth phase were taken and inoculated in a 96-well plate at a density of 5,000 cells/well, where 6 replicate wells were set for each group.
(2) The cells were cultivated for 24 h, then a compound was added at different concentrations, and the cells were further cultivated for 24 h. In groups 1 to 5, the compound 3 (20-O-VK) was added at concentrations of 0.1 μM, 1 μM, 2.5 μM, 5 μM, and 10 μM, respectively. A group C was a control group, and a same volume of a medium was added in the control group.
(3) After the 24 h of cultivation, a medium including the compound was removed, and then a DMEM medium including 10 μL of CCK-8 was added to each well.
(4) The plate was incubated in a 5% CO₂ and 37° C. incubator for 2 h.
(5) The plate was shaken at room temperature for 15 s, and then the absorbance (OD) of each well was determined at 450 nm with a microplate reader.

In this example, mouse neuroblastoma-2a (N2a) cells stably expressing human APP695 could stably produce Aβ, served as an in vitro AD screening model, and was provided by American Type Culture Collection (ATCC).

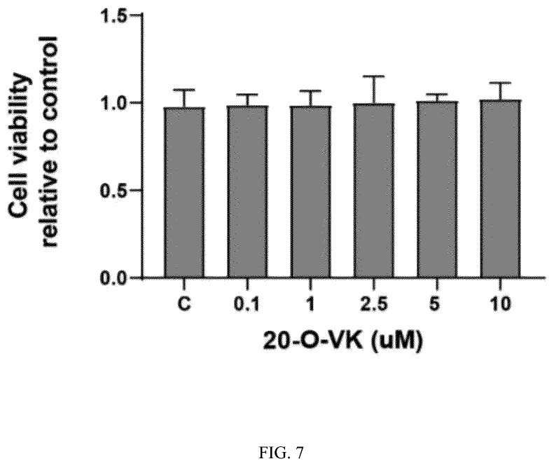

Detection results were shown in FIG. 7. The results show that the C21 steroidal saponin does not have a significant impact on the proliferation of N2a-APP695 cells. Viabilities of N2a-APP695 cells treated with the C21 steroidal saponin and not treated with a drug were determined by the CCK-8 method, and after the 24 h treatment, there was no significant difference in a cell viability between the control group and the C21 steroidal saponin treatment group (p>0.05). These results show that the C21 steroidal saponin does not have significant cytotoxicity against N2a-APP695 cells.

### Example 6 Impacts of a C21 Steroidal Saponin on the Expression of Neuroblastoma Cell-Associated Proteins

(1) N2a-APP695 cells in a logarithmic growth phase were taken and inoculated in a 6-well plate at a density of 205 cells/well.
(2) The cells were cultivated for 24 h, then a compound was added at different concentrations, and the cells were further cultivated for a corresponding period of time. In groups 1 to 3, the compound 3 (20-O-VK) was added at concentrations of 1 μM, 5 μM, and M, respectively. A group C was a control group, and a same volume of a medium was added in the control group.
(3) Cells were collected, proteins were extracted, and WB assay was conducted. Antibodies used were: beta Amyloid Polyclonal Antibody (CT695, Invitrogen, USA), BACE1 (#5606, CST, USA), SQSTM1/p62 (#5114S, CST, USA), LC3B (#2775S, CST, USA), Beclin 1 (#3495T, CST, USA), and GAPDH (#5174S, CST, USA).

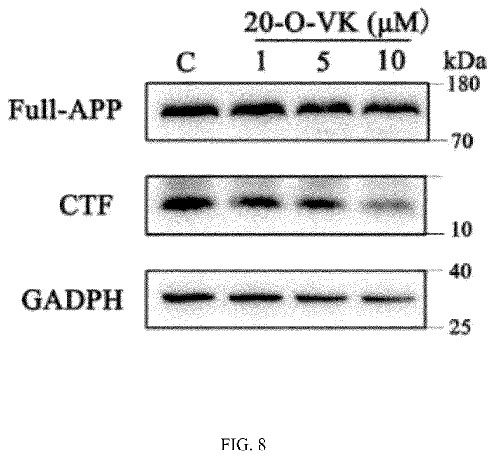

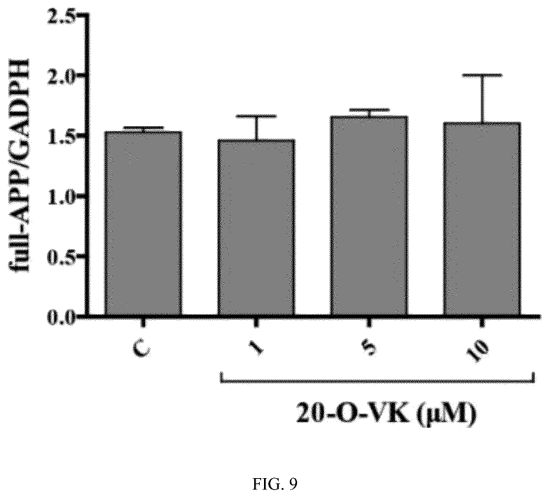

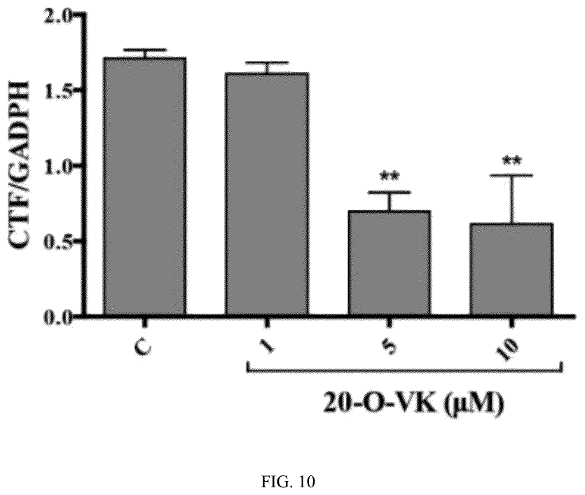

The impacts of the C21 steroidal saponin on full-APP and CTF were first detected, and results were shown in FIG. 8 to FIG. 10. The results show that the C21 steroidal saponin has an inhibitory effect on the expression of a CTF protein in N2a-APP695 cells, but does not affect the expression of a full-APP protein. Expression levels of the full-APP and CTF proteins in N2a-APP695 cells treated with the C21 steroidal saponin and not treated with a drug were determined by WB. After the 24 h treatment, compared with the control group, expression levels of the CTF protein in the 5 μM and 10 μM C21 steroidal saponin treatment groups significantly decreased (p<0.01), but an expression level of the full-APP protein did not significantly change (p>0.05). These results show that the C21 steroidal saponin at concentrations of 1 μM, 5 μM, and 10 μM can reduce the expression of the CTF protein in a concentration-dependent manner.

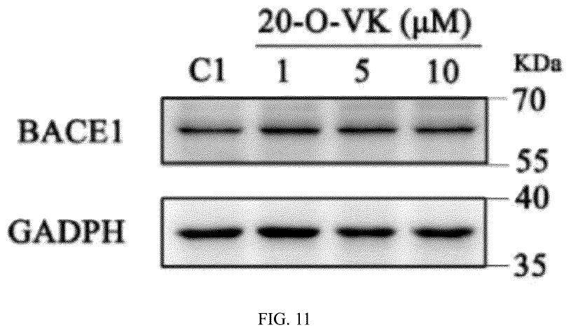

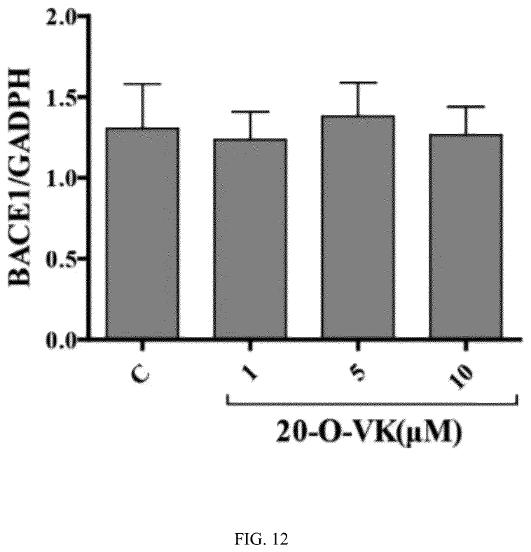

Further, an expression level of a BACE1 protein in N2a-APP695 cells was detected, and results (FIG. 11 and FIG. 12) indicated that, after the N2a-APP695 cells were treated with the C21 steroidal saponin for 24 h, the expression of the BACE1 protein did not change significantly (p>0.05). These results show that the C21 steroidal saponin may not reduce an expression level of CTF by blocking the upstream protein BACE1 for producing CTF.

In order to clarify a mechanism of the C21 steroidal saponin to inhibit the CTF protein in N2a-APP695 cells, Expression levels of the autophagy-associated protein LC3B II, the autophagy substrate protein P62, and the Beclin 1 in N2a-APP695 cells after 3 h, 6 h, 12 h, and 24 h of the treatment with the C21 steroidal saponin were determined by WB, and expression levels of the APP-full and CTF proteins in N2a-APP695 cells after 3 h, 6 h, 12 h, and 24 h of the treatment with the C21 steroidal saponin were investigated.

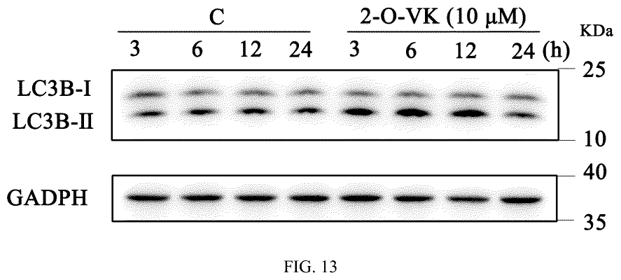

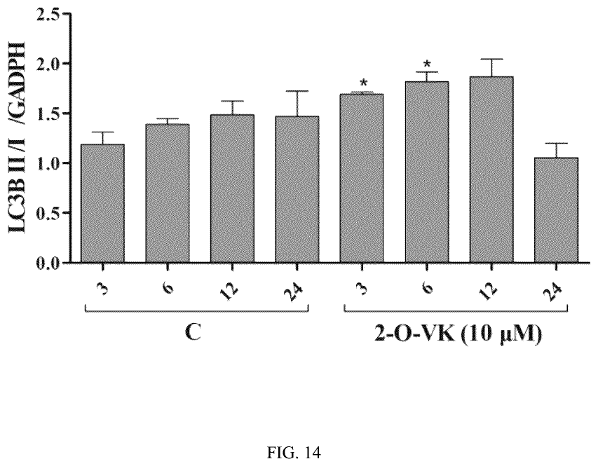

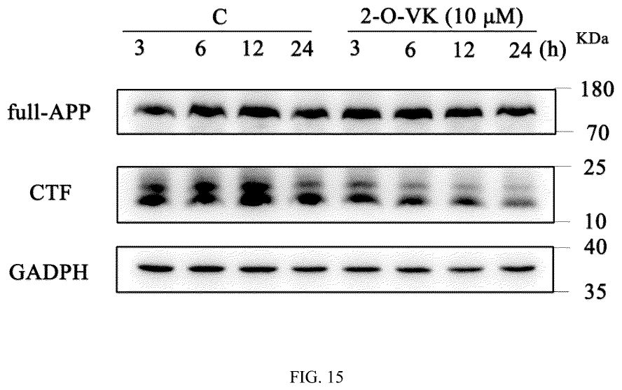

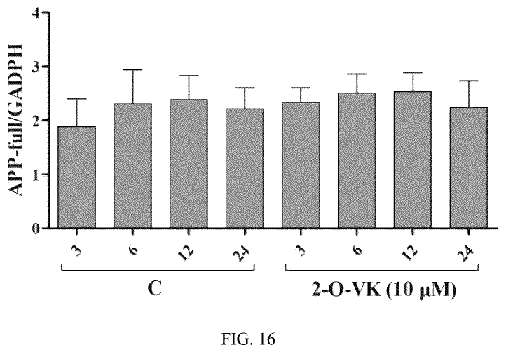

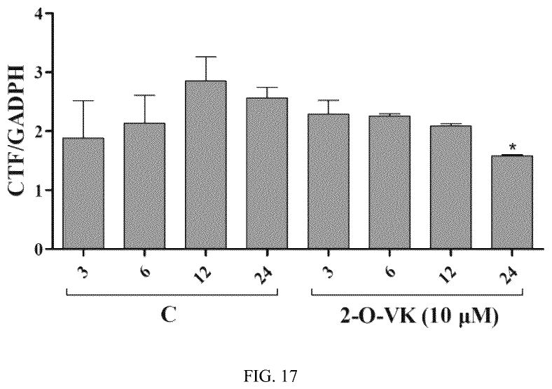

Results were shown in FIG. 13 to FIG. 17. Results of pairwise comparison by the LSD-t test show that, at 3 h and 6 h after administration, compared with the control group, the expression levels of LC3B II/LC3B I in the 10 μM 20-O-VK group significantly increase (p<0.05), indicating that the clearance of CTF in N2a-APP695 cells by 20-O-VK at 3 h and 6 h after administration may be allowed by enhancing the autophagy (as shown in FIG. 13 and FIG. 14). 3 h, 6 h, 12 h, and 24 h after the administration of 20-O-VK (10 μM), the expression of the APP-full protein does not change significantly. At 3 h, 6 h, and 12 h after the administration, the expression level of the CTF protein tends to decrease in a dose-dependent manner, and at 24 h after the administration, the expression level of the CTF protein decreases significantly (p<0.05). However, at 24 h after the administration, the expression levels of LC3B II/LC3B I tend to decrease (as shown in FIG. 15 to FIG. 17). It can be known that an autophagy level decreases after the decrease of the expression of the CTF protein at 24 h, indicating that the decrease of the expression of the CTF protein may be related to intracellular autophagy.

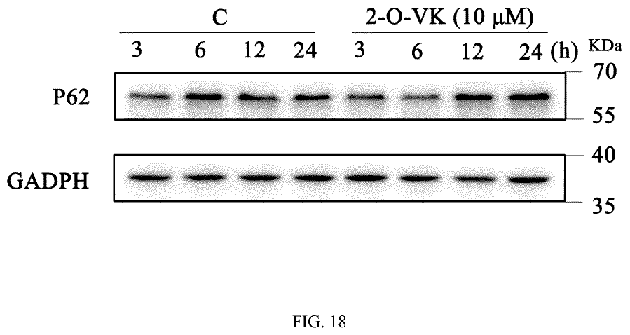

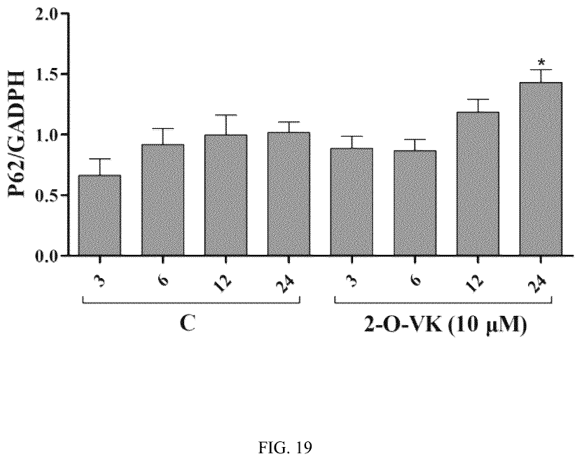

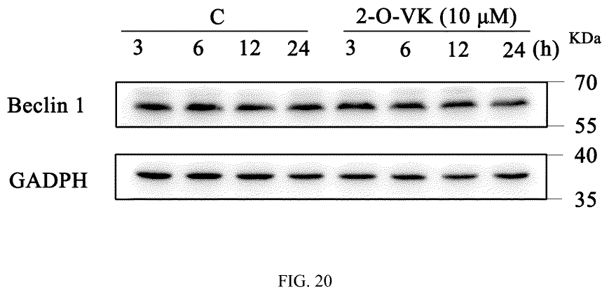

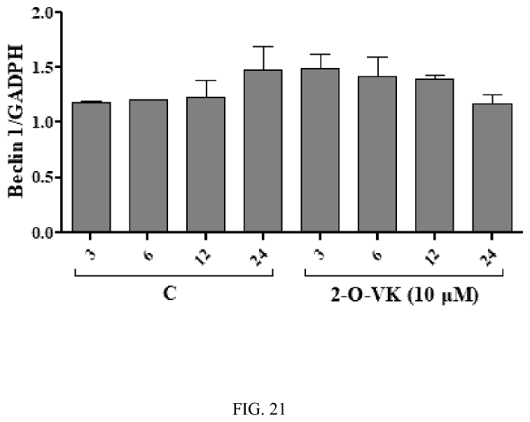

In order to further clarify that the decrease of the expression of the CTF protein in N2a-APP695 cells after the administration is related to autophagy, the expression levels of the autophagy-associated proteins P62 and Beclin 1 were investigated, and results were shown in FIG. 18 to FIG. 21. The results show that an expression level of the autolysosome substrate P62 tends to decrease at 6 h after the administration and significantly increases at 24 h after the administration, while an expression level of the protein Beclin1 tends to increase at 3 h, 6 h, and 12 h after the administration and tends to decrease at 24 h after the administration. Dynamic changes of the autophagy-associated proteins LC3B II/LC3B I, P62, and Beclin 1 further indicate that the reduction of the CTF protein in N2a-APP695 cells by 20-O-VK may be allowed by regulating autophagolysosomes.

In summary, the C21 steroidal saponin, an active ingredient in the pharmaceutical composition provided in the present disclosure, can significantly reduce the production of Aβ in N2a-APP695 cells, increase a clearance rate of excess Aβ, promote the proliferation of neuronal cells, and have a protective effect for neurotransmitter (such as glutamate)-induced neuronal cytotoxicity caused by the excessive accumulation of Aβ, thereby comprehensively playing a role of treating a neurodegenerative disease. Therefore, the pharmaceutical composition has a promising application prospect in the preparation of Aβ-formation inhibitors and neuroprotective agents and drugs for preventing or treating neurodegenerative diseases such as AD, Parkinson's disease, and Huntington's disease.

The analytical methods involved in the present disclosure are specifically introduced through the above specific embodiments. It should be noted that the above introduction is merely intended to help those skilled in the art well understand the methods and ideas of the present disclosure, and rather than to limit the relevant contents. Those skilled in the art may make appropriate adjustments or modifications to the present disclosure without departing from the principle of the present disclosure, and such adjustments and modifications shall also fall within the protection scope of the present disclosure.

What is claimed is:

1. A pharmaceutical composition for preventing and/or treating a neurodegenerative disease, comprising one or more selected from the group consisting of a C21 steroidal saponin, a pharmaceutically-acceptable salt of the C21 steroidal saponin, and a solvate of the C21 steroidal saponin and a pharmaceutically-acceptable carrier, wherein the C21 steroidal saponin has a structure shown in the following formula I: 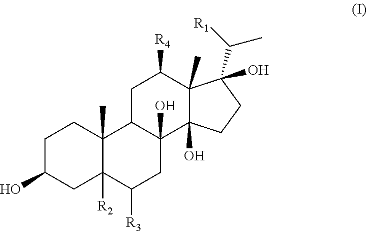
wherein R₁ is one or more selected from the group consisting of H, O, hydroxyl, optionally-substituted C₁-C₄ alkyl, optionally-substituted C₁-C₄ alkoxy, and 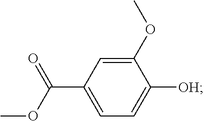
R₂ is one or more selected from the group consisting of H, O, hydroxyl, optionally-substituted C₁-C₄ alkyl, and optionally-substituted C₁-C₄ alkoxy;
R₃ is one or more selected from the group consisting of H, O, hydroxyl, optionally-substituted C₁-C₄ alkyl, and optionally-substituted C₁-C₄ alkoxy;
R₄ is one or more selected from the group consisting of H, O, hydroxyl, optionally-substituted C₁-C₄ alkyl, optionally-substituted C₁-C₄ alkoxy, 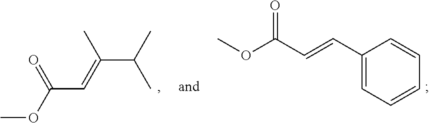
and
adjacent carbon atoms in a multi-membered ring of a compound with the structure shown in the formula I are linked through a single, double, or triple bond.

2. The pharmaceutical composition according to claim 1, wherein the neurodegenerative disease comprises one or more selected from the group consisting of dementia, Parkinson's disease, and Huntington's disease.

3. The pharmaceutical composition according to claim 2, wherein the dementia comprises one or more selected from the group consisting of Alzheimer's disease (AD), vascular dementia, Lewy body dementia, and frontotemporal dementia.

4. The pharmaceutical composition according to claim 1, wherein the pharmaceutically-acceptable carrier comprises one or more selected from the group consisting of a filler, an adhesive, a disintegrant, a solvent, a preservative, a lubricant, and a corrigent.

5. The pharmaceutical composition according to claim 1, wherein the C21 steroidal saponin is one or more selected from the group consisting of compounds 1 to 5 shown in the following structures:

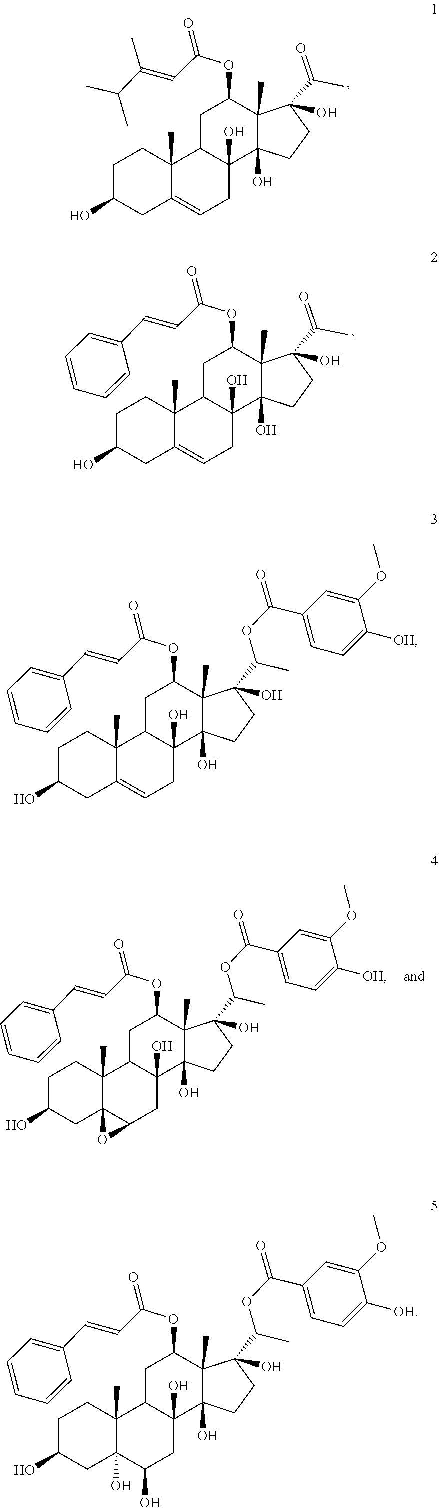

6. A use of one or more selected from the group consisting of a C21 steroidal saponin, a pharmaceutically-acceptable salt of the C21 steroidal saponin, and a solvate of the C21 steroidal saponin in preparation of a product for preventing and/or treating a neurodegenerative disease, wherein the C21 steroidal saponin has a structure shown in the following formula I: 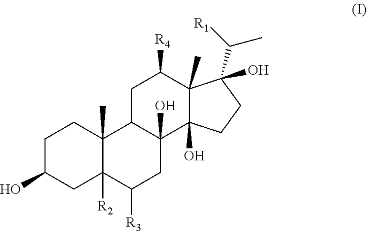
wherein R₁ is one or more selected from the group consisting of H, O, hydroxyl, optionally-substituted C₁-C₄ alkyl, optionally-substituted C₁-C₄ alkoxy, and 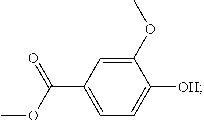
R₂ is one or more selected from the group consisting of H, O, hydroxyl, optionally-substituted C₁-C₄ alkyl, and optionally-substituted C₁-C₄ alkoxy;
R₃ is one or more selected from the group consisting of H, O, hydroxyl, optionally-substituted C₁-C₄ alkyl, and optionally-substituted C₁-C₄ alkoxy;
R₄ is one or more selected from the group consisting of H, O, hydroxyl, optionally-substituted C₁-C₄ alkyl, optionally-substituted C₁-C₄ alkoxy, 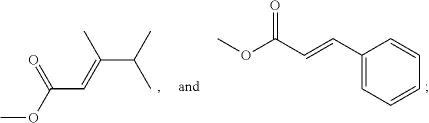
and
adjacent carbon atoms in a multi-membered ring of a compound with the structure shown in the formula I are linked through a single, double, or triple bond.

7. The use according to claim 6, wherein the neurodegenerative disease comprises one or more selected from the group consisting of dementia, Parkinson's disease, and Huntington's disease.

8. The use according to claim 7, wherein the dementia comprises one or more selected from the group consisting of AD, vascular dementia, Lewy body dementia, and frontotemporal dementia.

9. The use according to claim 6, wherein the product comprises one or more selected from the group consisting of a drug, a health care product, and a food.

10. The use according to claim 6, wherein the C21 steroidal saponin is one or more selected from the group consisting of compounds 1 to 5 shown in the following structures: [chemical structure]
